(19) United States
(12) Patent Application Publication
(10) Pub. No.: US 2007/0082411 A1
(43) Pub. Date: Apr. 12, 2007

(54) Detection and identification of biological materials using functionalized quantum dots

(76) Inventor: James Johan Muys, Christchurch (NZ)

Correspondence Address: JAMES J. MUYS, 27 B DELL AVENUE, REMUERA, AUCKLAND, 1005, NZ

(21) Appl. No.: 11/415,681

(22) Filed: May 2, 2006

Related U.S. Application Data
(60) Provisional application No. 60/724,211, filed on Oct. 7, 2005.

(51) Int. Cl. G01N 33/551

(52) U.S. Cl. 436/524

(57) ABSTRACT

A method and apparatus has been invented to detect and identify biological materials and their properties based on the affinity of water-soluble, semiconductor nanocrystals bioconjugates. A plurality of nanocrystals comprise quantum dots of varying sizes functionalized with one or more material or compound, where one size left un-conjugated, are contacted in solution with biological materials from a sample and then separated using a filter. The plurality of quantum dots act as a test to detect the presence or absence of a target material in a given sample, with those having an affinity remaining bound to the biological materials and subsequently trapping in the filter. The quantum dot bioconjugates form functional particles and may be conjugated with several layers, such as primary and secondary antibodies. An emitter laser or lamp is used to activate the quantum dots. The biological material is captured and immobilized in a filter which is formed in a connectable apparatus, which is inverted to employ the inventive methods. A detector is used to detect the fluorescence emitted from each size of quantum dots present that may, or may not, be tagged to the biological materials captured in the filter.

### CROSS-REFERENCE TO RELATED APPLICATIONS

This application claims the benefit of provisional patent application Ser. No. 60/724,211, filed 2005 Oct. 7 by the present inventor.

### FEDERALLY SPONSORED RESEARCH

Not Applicable

### BACKGROUND OF THE INVENTION—FIELD OF INVENTION

The field of the invention relates to the biological applications of a composition comprising of water-soluble semiconductor fluorescent nanocrystals. More specifically, this invention includes a method and apparatus for detecting, separating and capturing a solution of functional and non-functional nanocrystals based on their affinity to a biological material in which the nanocrystals are employed.

### BACKGROUND OF THE INVENTION—PRIOR ART

In biology it is of interest to mark structures such as cells or viruses with fluorescent materials for accurate identification, ease of detection and microscopic analysis. Traditionally, organic dye fluorophores have been the favored materials and have the capability to be modified with a range of materials, enabling targetted binding to a wide range of biological structures based on known affinties and chemistries. Upon binding of the dye to the target biological material, an activation light of a given wavelength is used to excite the dye, from which it responds by fluorescently emitting a characteristic light radiation specific to the properties of the organic dye employed. However, traditional organic dyes have numerous limitations when used to tag biological materials.

Semiconductor fluorescent nanocrystals (“quantum dots”) are nanometer sized semiconductor, light-emitting crystals, spherical in shape and have superior fluorescent properties to organic dyes. Quantum dots are generally synthesized with Type II-VI (e.g. CdSe, CdTe, CdS and ZnSe) or Type III-V (e.g. InP and InAs) column elements from the periodic table and can be capped with numerous shells, layers or molecules to modify their physical properties, such as for surface functionalization (Chan et al., 2002, Curr. Opin. Biotech. 13:40-46). Integration of quantum dots in biology was achieved in breakthroughs showing that highly luminescent quantum dots could be made water-soluble and subsequently biocompatible using surface modification techniques such as silica/siloxane coatings (Gerions, 2001, J. Phys. Chem. B 105:8861-8871; and Bruchez et al., 1998, Science 281:2012-2015) or direct absorbtion of bifunctional ligands (Chan et al., 1998, Science 281:2016-2018), which presented them useful tools in biology. Quantum dots are emerging as the new biological label with applications and properties superior to traditional fluorescent proteins and organic dyes (Watson et al., 2003, Biotechniq. 34:296-300; Michalet et al., 2005, Science 307(5709):538-544; and Chan et al., 2002, Biotechnol. 13:40-46).

Most of the limitations with traditional organic dyes are a result of the extremely limited absorptive and emissive capabilities. The first shortcoming is that the peak emission of organic dyes cannot be altered—each dye corresponds to a different molecule with a different pre-set emission wavelength, or fluorescent color, that is set by nature. Therefore, applications that make use of light frequencies that do not correspond to the emission peaks of pre-existing organic dyes cannot be performed. The second shortcoming is the narrow absorption pattern of organic dyes—dyes tend to display absorption peaks that are not always in convenient regions of the spectrum, making the excitation of various organic dyes challenging and costly. The third shortcoming is that of uneven absorption and emission peaks—organic dyes have a tendency to produce “shoulders” in the geometry of their emission and absorption peaks, which is a major disadvantage in applications that require Gaussian type emission patterns to work correctly. An additional shortcoming is that of stability—the lifetime of organic dyes varies but is generally low relative to that of other tagging mechanisms and organic dye fluorescence is controlled entirely by the molecular bonding properties of each individual dye. Finally, incident radiation absorbed by an organic dye molecule moves electrons into excited states, whereupon they decay and release light radiation. This emission cannot be altered because it corresponds to pre-set excited states of the dye molecule that are inherent to every molecule of that type.

Whereas the light emission ranges and possible forms of organic dyes are very limited, quantum dots can be made to emit light at any wavelength in the visible and infrared ranges, and can be inserted almost anywhere, including in liquid solutions, dyes, paints, epoxies, and sol-gels. Furthermore, quantum dots can be attached to a variety of surface ligands, and inserted into a variety of organisms in vivo (Dubertret, 2002, Science 298:1759-1762; and Larson et al., 2003, Science 300:1434-1436) or in vitro (Mansson et al., 2004, Biochem. Biophys. Res. Commun. 6; 314(2):529-34).

Recently it has been shown that functional quantum dots can be linked with biological molecules such as proteins (Mattoussi et al., 2000, J. Am. Chem. Soc. 122:12142-12150), DNA (Mitchell et al., 1999, J. Am. Chem. Soc. 121:8122-8123), peptides (Whaley et al., 2000, Nature 405:665-668) and nucleic acids (Niemeyer, 2001, Angew. Chem. Int. Ed. 40:4128-4158), with the potential to conjugate multiple biological molecules to a single quantum dot (Akerman et al., 2002, Proc. Natl. Acad. Sci. U.S.A. 99:12617-12621).

Numerous methods exist for covalently linking biological molecules to quantum dots to create a bio-molecular conjugates (“bioconjugate”) or functional quantum dot (Goldman et al., 2002, J. Am. Chem. Soc. 124:6378-6382; Jaiswal et al., 2004, Nature Methods 1:1; Mattoussi et al., 2000, J. Am. Chem. Soc. 122:12142-12150; and U.S. Pat. No. 6,114,038 to Castro (2000); U.S. Pat. No. 6,855,551 (2005) to Bawendi et al.; and U.S. Pat. No. 6,468,808 to Nie (2002)), which are used in labeling, detection and imaging applications to attach or bind a quantum dot to a biological material based on specific chemical or biological affinity. These methods employ a variety of chemistries to water-soluble quantum dots from which several cross-linker molecules can be coupled to enable the attachment of the primary functional biomaterial. Other examples of bioconjugate techniques enabling the attachment of various materials to quantum dots are known to those skilled in the art, refer, for example to; Bioconjugate Techniques (Academic Press, New York (1996)) and (Bailey et al., 2004, Physica E 25:1-12).

Generally, bioconjugation methods are classified into mechanisms using (Chan et al., 2002, Curr. Op. Biotech. 13:40-46): (1) Biofunctional linkages (Chan et al., 1998, Science 282:2016-2018), (2) Electrostatic attraction (Mattoussi et al., 2000, J. Am. Chem. Soc. 122:12142-12150), (3) Hydrophobic attraction, (4) Silanization (Bruchez et al., 1998, Science 281:2013-2015), and (5) Nanobead linkages (Han et al., 2001, Nat. Biotech. 19:631-635). Examples of methods employing bioconjugative techniques are polyethylglycol modification of the underlying carboxyl quantum dots, and optimization of the surface loading of amino groups for high conjugation efficiency and specificity. Another example is modifying the quantum dots with peptides through the amino or carboxyl groups at the terminus, or using other residues, small molecules, proteins, or nucleic acids, and other methods known to those skilled in the art. More specifically, schemes used for the conjugation of antibodies to quantum dots are based on well-known chemistries using the fast and efficient coupling of thiols to maleimide groups, with reactive groups such as primary amines, alcohols, carboxylic acids and thiols used to link the antibodies to the quantum dots.

Quantum dots represent a marked increase in performance over standard organic dyes, because they can be tuned to absorb or emit at any visible or infrared wavelength and can be fabricated into a great variety of forms and media, eliminating completely the shortcomings of dyes. These unique abilities are due to their very small sizes (typically between 1-10 nm in diameter). At these sizes, quantum mechanics allow semiconductor materials to take on all new traits, including that of a bandgap that can be tuned with the addition or subtraction of only a few atoms to the quantum dot. The small size and its direct relationship to fluorescence also allows for incredible versatility and flexibility of form, letting phosphors match whatever shape their underlying light-emitting diode (LED) needs to assume.

When light impinges on quantum dots, it encounters discretized energy bands specific to the quantum dot. The discretized nature of quantum dot bands means that the energy separation between the valence and conduction bands (the bandgap) can be altered with the addition or the subtraction of just one atom—making for a size dependent bandgap. Pre-determining the size of the quantum dots fixes the emitted photon wavelength at the appropriate customer-specified color, even if it is not naturally occurring—an ability limited only of quantum dots. In addition, the extremely small size and versatility of quantum dots allows them to be inserted into any medium necessary to accommodate research.

While fluorescent emissions from functional quantum dot bioconjugates have been used to detect the presence or absence of a target substrate in a sample, U.S. Pat. No. 6,114,038 to Castro (2000), at present there remains no effective method and apparatus for employing a plurality of differently functional and non-functional quantum dots and separating unbound quantum dots those remaining bound to the biological material.

### BACKGROUND OF THE INVENTION—OBJECTS AND ADVANTAGES

When compared to traditional organic fluorophore dyes (Haugland, R. P. Handbook of Fluorescent Probes and Research Products (Molecular Probes, Eugene, Oreg., U.S.A, 2002) or fluorescent proteins, quantum dots have distinctive optical and spectral properties that provide several unique advantages for fluorescent tagging of biological materials. These advantages are well known and traditional organic dyes suffer from several problems, such as photobleaching, spectral cross-talking and narrow excitation. quantum dots have the potentional to overcome these problems” (Xiaohu, 2003, Tren. Biotech. 21:9). Generally, quantum dots are broadly compared with organic dyes as being superior with respect to (Alivisatos, 1996, Science 271:933-937); composition and size dependent tunable emission wavelengths; large absorptions cross sections; wide absorption profiles; good photostability; and narrow emission spectra.

Firstly, quantum dots have superior fluorescent properties, which allow tunable emission of the peak wavelengths by simply changing the composition of the underlying semiconductor crystal. Currently, numerous sized quantum dots are available with distinct peak emissions ranging from 300-850 nm. In addition, quantum dots have a narrow bandwidth (full width half max) less than 30 nm, and the Gaussian, or bell-shape spectral emission characteristics give a neat and predictable spectrum, which is centered at a peak. Excitation of quantum dots is easy due to their activation by a single light wavelength, which results in a high quantum yield and descretely detectable luminescent emission peaks. The broadband absorption properties of quantum dots make simultaneous activation advantegous in systems employing several sized quantum dots, and the Stokes shift means that in the visible spectral region there is a shift of 15 nm between the emission and absorption wavelenghts. This large Stokes shift allows the quantum dot emission signals to be separated and distinguishable from background fluorescence, a property not done easily with conventional dyes (Yang et al., 2000, Proc. Natl. Acad. Sci. U.S.A. 97:1206-1211). Furthermore, quantum dot activation can be achieved using light sources shorter than the emission wavelengths of the quantum dots and thus is effectively independent from excitation source.

Moreover, quantum dots have an incredible hight intesity and brightness, large quantum yieds and wide spectral absortion cross-sections, presently the possibility of longer integration times and lifetime characterisitcs. Furthermore, as they are derived from inorganic particles, they are more photostable than traditional organic fluorophores and last an order of magnitude longer at intense fluorescence—their fluorescent lifetimes are many times greater than organic dye lifetime and have been known to luminesce with no photodegredation over a period of 2 hours.

Finally, as quantum dots are inorganic semiconductor nanocrystals it is possible to visualize them using electron microscopy and functionalization of quantum dots are easily performed using stable bioconjugative techniques known to those skilled in the art, which have no effect of their underlying optical or electronic properties. Due to their similarity to bio-macromolecules, bioconjugated quantum dots are well suited as contrasting agents for molecular imaging and detection applications.

Accordingly, from the discussion of the superior advantages of using quantum dots over traditional dyes, it is an object of this invention to provide a better method for detecting and identifying biological materials using fluorescence radiation emitted from quantum dot bioconjugates; this goes some way to overcoming the above disadvantages, or which at least provides a useful choice over existing approaches. In particular, the present invention seeks to use a filter separation apparatus for separating unbound functional quantum dot bioconjugates and non-functional quantum dots from those bound to biological materials. Accordingly, it is yet another advantage of this invention to simultaneously employ and detect a plurality of differently functionalized bioconjugated and non-functional quantum dots for determining the properties of biological material present in a sample.

### SUMMARY OF THE INVENTION

The present invention provides a method and apparatus for simultaneously performing multiple tests on a biological sample by separating a plurality of different sized, water-soluble quantum dot bioconjugates bound to biological materials present in a sample from those remaining unbound. Comprised in this invention is a filter for trapping the biological material from a sample mixed in a solution consisting of functional quantum dot bioconjugates and non-functional quantum dots. A detector is used to detect the distinguishing fluorescent wavelengths emitted from the sizes of quantum dots present in the solution. Moreover, the functional quantum dots in the solution will be conjugated with a variety of biomaterials allowing for the simultaneous detection and analysis of the biological material from a sample based on affinity chemical and biological binding and interaction with the quantum dot bioconjugates.

### BRIEF DESCRIPTION OF THE DRAWINGS

FIG. 1 shows steps illustrating the methods used in the invention.

FIGS. 2A to C descriptively illustrates the mix-and-flip separation apparatus.

FIGS. 3A to D sequentially illustrates the operation of the mix-and-flip apparatus system.

FIGS. 4A to B illustrates the addition of the suction outlet to the mix-and-flip apparatus.

FIGS. 5A to E depicts the operation of the mix-and-flip apparatus using suction-driven filtration.

### DRAWINGS—REFERENCE NUMERALS

| | |
|---|---|
| 210 | base tube |
| 212 | filter tube |
| 214 | connector |
| 216 | mesh filter |
| 218 | nozzle |
| 220 | base tube open-end |
| 222 | base tube closed-end |
| 310 | 7 nm functional quantum dots |
| 312 | 6 nm non-functional quantum dots |
| 314 | 5 nm functional quantum dots |
| 316 | mammalian cells |
| 318 | light source |
| 320 | detector |
| 322 | fiber optical cable |
| 324 | solution |
| 410 | suction outlet |
| 412 | plastic tube |
| 414 | pump |
| 416 | filter-output tube |
| 418 | pore filter |
| 510 | low-pass light filter |
| 512 | band-pass light filter |
| 514 | 10 nm functional quantum dots |
| 516 | 9 nm functional quantum dots |
| 518 | 7 nm functional quantum dots |
| 520 | 8 nm non-functional quantum dots |
| 522 | viral spores |

### DETAILED DESCRIPTION OF THE INVENTION

The present invention provides a method and apparatus for accurately detecting the properties of biological materials present in a sample in vitro. The present invention is premised on the advantageous discovery a cocktail solution comprising of a plurality differential water-soluble quantum dot bioconjugates can be functionalized and used as an in vitro assay, revealing information on the characteristics of the biological material present in the sample from the basis of the known properties of the functional material conjugated to the quantum dots. The present invention is used to determine the properties of biological material present in the sample based on whether the functional material conjugated to the quantum dots, has a recognized affinity known to those skilled in the art, and is attracted to the biological material. Using several functional quantum dots conjugated with different biomaterials, enables a method for simultaneously deducing the properties of the biological sample. The present invention is also premised on the discovery that those quantum dot bioconjugates bound to the biological materials can conveniently be separated from those non-functional quantum dots and quantum dot bioconjugates not bound to the biological material in the sample using a filter. The present invention is combined to form an apparatus for rapid and accurate detection of the various quantum dot sizes present in the cocktail solution using their distinguishing waveforms.

The term “biological material” includes any biological material that is capable of self-replication either directly or indirectly. Representative examples include bacteria, DNA, proteins, fungi- including yeast, algae, protozoa, eukaryotic cells, cell lines, hybridomas, plasmids, viruses, plant tissue cells, lichens and seeds. Materials such viruses, vectors, cell organelles and other non-living material existing in and reproducible from a living cell may be deposited by deposit of the host cell capable of reproducing the non-living material are also included in this definition. Examples of biological materials formed from the sample include specimens taken from a patient, such as a malignant tumor biopsy, and are often highly heterogeneous and contain different cell populations in various microenvironments.

The term “bioconjugate” as used herein refers to methodologies and techniques used to attach functional materials, such as biomolecules to the water-soluble quantum dots, done using any stable physical or chemical associations known by those skilled in the art. Typically, such methods utilize the hydrophilic attachment groups of the water soluble quantum dot directly or indirectly with any mean suitable. If the biomolecules are attached indirectly, linker molecules are often used to form intermediary bonds between the quantum dot and biological molecules. For more information on bioconjugative techniques that can be used to attach biomolecules to quantum dots, known to those skilled in the art refer to Bioconjugative Techniques (Academic Press, New York, (1996)).

The term “functional” as used herein refers to the quantum dot being bioconjugated with biomaterials.

The term “non-functional” as used herein refers the quantum dots not being conjugated with biomaterials. More specifically, non-functional quantum dots are used to distinguish from those functional quantum dot bioconjugates present in the cocktail solution, which have been modified with biomaterials designed to bind or attach to the biological material present in the sample.

The term “cocktail solution” as used herein refers to a colloidal, water-based solution comprising of an assortment of quantum dot bioconjugates of different sizes with different functional materials, with at least one size of quantum dot present in the solution acting as a control and remaining non-functional, or not conjugated with a biomaterial intended to bind to the biological materials present in the sample.

In view of the above, the present invention provides in one embodiment a plurality of functional, water-soluble quantum dot bioconjugates. As the fluorescence properties of quantum dots are largely size dependent it is an object of this invention to use multiple differently sized quantum dot bioconjugates.

By functionalizing the quantum dots where the emission wavelength of those quantum dots are known and using detectors whose detection range is within that range, the set of quantum dots can be detected if the emission range of the other dots do not substantially overlap each other. It is advantageous to use quantum dots due to their narrow emission bandwidth, which can be directly controlled by changing their size. This allows the detection of quantum dots of different sizes in a mixed solution by their known peak emission frequency characteristics. Several terms are used interchangeably referring to the emission of light by a quantum dot following stimulation with light of another color, they are; emission, luminescence, photo-luminescence and fluorescence. Similarly, several terms used to describe the capture of light by the quantum dot, these are; absorbance, absorption, excitation and photo-excitation.

Accordingly, a preferred embodiment of the present invention provides a solution comprising of a plurality of water-soluble quantum dots functionalized using bioconjugation techniques, known by those skilled in the art, to the form a cocktail solution, where at least one of the sizes of quantum dots present in the solution remain non-functional. The assortment of functional quantum dots combined in the solution are those designed to detect a specific biological material type or class or a biological material in a specific state or condition, which by combining different quantum dot bioconjugates into a single solution would be useful either in testing, analysis, diagnosis or for detection. The size differentiation of the quantum dots used in the cocktail solution are variable, such that each size has a peak emission wavelength, which is easily distinguishable from the fluorescent emission of the adjacent sized quantum dots present. Furthermore, in order to avoid large cross-over interference it will be advantageous to use quantum dots emitting within a narrow and predictable band range. This enables more quantum dots of different sizes to be included in the cocktail solution. As most biological systems are present in aqueous solutions, it is a preferred embodiment of this invention to utilize water-soluble quantum dots.

Another embodiment of the invention comprises of the addition of a supplement or combination of supplements to the cocktail solution, which when mixed with the biological sample is designed to modify or induce those biological materials into a specific state or form. Supplements include chemicals and biological reagents; examples are those with a stimulation or digestion effect, such as protein digesting enzymes and proteases like trypsin; collegenase and pronase for digestion and disassociation; fixation chemicals such as glutaraldehyde; stimulation and inhibition specific chemicals; and other chemicals and biological reagents known to those skilled in the art.

The present invention also provides a method for separating a solution consisting of a plurality of quantum dot bioconjugates and biological materials into two separate components comprising of—(1) biological materials present in a sample and those quantum dot bioconjugates bound to, and (2) another population of unbound quantum using a passive filter. Materials that can be used as the filter include; strainers, nylon wool, gauze, mesh, mesh baskets, gauze, membrane filters, porous membranes, nano-patterned fabricated membranes (e.g. Silicon nitride membranes with lithography patterned holes) known to those skilled in the art, or any mesh or passive filtration system capable of the fine filtration and separation of particles in fluid. Examples of the filter include porous membrane filters, such as isopore polycarbonate membranes and mixed cellulose ester membranes, which are commercially available from manufacturers such as Millipore Corporation U.S.A—whom supply a variety of filters from as small as 50 nm pores to as large as 80 μm mesh, enabling most biological materials, from as small as viruses or proteins, to as large as cellular tissue be captured in the filter.

Methods employing filters with low micrometer and nanometer pore or mesh sizes will require the solution to be assisted through using forces other than gravity, such as suction, centrifugation and hydrodynamic forces by pumping, micro-fluidics and electro-kinetic forces, known to those skilled in the art. It is a property of the inventive methods to incorporate an externally driven pump to assist in circumstances where gravity cannot force the solution effectively through the filter. The use of external forces are particularly needed in cases where the biological material components present in the sample are known, or suspected, to be of small sizes, such as DNA, viruses or proteins. This is due to the employment of a filter will smaller pore or mesh size necessary to capture the bulk of the biological material, and which will require forces greater than that of gravity for filtration.

In cases where there is a large quantity of biological material or where a solution contains biological of different sizes or types, it is a property of this invention to combine several filters, or different types of filters in series. Such an example would be if the sample consisted of several biological materials of different types and sizes and needed different pore sizes to be able to effectively and efficiently extract all the biological material from the solution. The properties of the filter will depend on the biological sample and determining factors include size, quantity and physical and chemical nature of the sample. Another embodiment of the inventive methods, which will become apparent from the description of the apparatus, includes placement of the filter at an angle with respect to the tube or device it is mounted in. The advantage of angling the filter is that a larger filter diameter can be placed inside the tube and hence, the methods of the invention may incorporate a larger quantity or volume of biological material in the sample due to the scalability of the filter.

Furthermore, it is a property of the invention to chemically or biologically modify the filter in order to prevent quantum dots that are not bound to the biological material present in the sample from binding as they pass through. This includes changing the hydrophobic or hydrophilic properties of the filter material or including a washing solution designed to rinse the excess fluid remaining in the filter after filtration of the solution. The properties of the filter can be modified in order to better capture and attach biological materials, such as using biological attachment enhancers like collagen and poly-1-lysine proteins.

The presence or absence of quantum dots from the filtered solution is detectable by exposure to a light, or lamp source designed to excite, or activate, the quantum dots to emitting a luminescent peak. Examples of possible sources that can be used to activate quantum dots to fluoresce are helium/neon lasers, green lasers, and argon lasers. Examples of lamps that can be used in the invention are ultraviolet (U.V), sodium, halogen, deuterium, tungsten, mercury, and xenon lamps. Intensity of these lamps and light sources can be modulated and controlled using slits, filters, irises, or pinholes and several lamps and light source can be used simultaneously.

Detection of quantum dots in this invention is achieved by spectrometry, flow cytometry, and microscopy methods and techniques using devices, such as optical detection systems like fluorescent microscopy, or electrically with photo-sensitive semiconductor devices. It is an object of this invention to use optical and electrical detection systems for simultaneously sensing the presence and intensity of different sized quantum dots. However, the advantage of electrical detection is in the miniaturization and systems integration with benefits particularly in automation. Typically, electrical systems share the common ability of detecting radiation in the form of the photons emitted and output an electronic signal based on the intensity of the radiation. Accordingly, the photosensitive devices used in this invention will be able to effectively and simultaneously detect the different radiation wavelengths emitted from the different sized quantum dots. Due to the low levels of fluorescent light emitted by the quantum dots, the device will be capable of detecting low levels of radiation.

Spectrometers are a family of photosensitive devices, which include spectrophotometers, fluorescent spectrometers and fluorometers. The spectrometer used as the detector in this invention will be capable of sensing small amounts of radiation emitted by the quantum dots in the extreme-U.V to the near infrared (n-IR) light wavelengths (200-1100 nm) with high accuracy. The detector will have the ability to detect a narrow range or band of light wavelengths. The advantages of using such devices as silicon photodiodes are their ability to detect extremely minute quantities of light below 1 pW/cm2, such types of silicon photo-detectors include, planar diffused silicon photodiodes, which are available in multi-elemental forms.

Multi-element array photo-detectors are multi-channel arrays consisting of a number of single element photodiodes, which are laid adjacent to each other to form a one-dimensional sensing area on a common cathode substrate. This enables the simultaneous measurement of a source of several wavelengths. Furthermore, this offers a low cost alternative when a large number of detectors are required. Examples of photo-detectors used in this invention include charge-coupled device (CCD) sensors, known to those skilled the art. Examples of CCD sensors are; Sony ILX511, Hamamatsu S9840, and Toshiba TCD 1304AP, which can be coated with thin-films of materials such as phosphor in order to increase their sensitivity in a specific light range. Typically, these devices share the common ability of detecting radiation in the form of the photons and output an electronic signal based on the relative intensity of the radiation. Ideally the detector will closely match the radiation bands emitted by the variable sized quantum dots and will be capable of sensing low levels of light in the extreme-U.V (˜200 nm), U.V (˜250 nm), Visible (˜800 nm) and n-I.R (˜1100 nm) light ranges.

As the fluorescent radiation emitted by quantum dots is relatively low, a very sensitive device capable of detecting this radiation is needed, and it is an object to employ other devices capable of detecting and amplifying wavelengths to the detector such as photo-multipliers. Other devices used in the invention include optical-electronic or semiconductor devices, which improve or enhance the detection of fluorescent light. Additionally, gratings with varying slit widths, as well as mirrors and collimators can be used to deflect, direct or split the emitted light onto the detector. Other devices included in this invention are common spectrometer components known to those skilled in the art, such as beam splitter devices, mirrors and filters.

It is an object of this invention to employ devices capable of applying an excitation light to, and detecting a plurality of fluorescent emissions from the activated quantum dots. Light emissions from the quantum dots will form several distinct wavelength bands, determined by the number of quantum dot sizes present in the solution. Filters designed to purify the light sources will be used to avoid cross-contamination or interference with the fluorescent emission lights from the quantum dots. In addition, components such as optical fibers, using non-solarizing fibers suited for transmission of wavelengths in the range of 198 to 1200 nm will be used. Lenses for collecting the plurality of fluorescent emissions from the quantum dots and applying the activation light to the quantum dots may be included as passive devices used in the apparatus of the invention. For fluorescent detection, variable filters (e.g. long-pass, band-pass and high-pass) can be fitted to adjust the emission wavelengths, as well as filtering the quantum dot emissions from the activation light. Hence, light filters and mirrors can be incorporated into the invention and used to transmit only a certain wavelength range or redirect light onto photosensitive detectors, preventing the detector from detecting unwanted light from external sources, such as the emitter source or to reduce the light emissions in order to prevent light saturating the detector.

Due to the light intensities emitted from the quantum dots being relatively low, it is an object of this invention to enhance and focus their emissions onto the detector for more accurate sensing. Hence, techniques such as employing fiber optical cables are used to focus fluorescent emissions from the quantum dots precisely onto the sensing area on the detector. In order to establish whether quantum dots are indeed present based on signals produced from the detector, it is also an object to incorporate additional detectors positioned at various locations. Such an example would be positioning detectors at locations prime for detecting light from the quantum dots bound to biological materials, which have been captured in the filter, as well as placing detectors primed to detect the presence of quantum dots in the filtered solution.

Accordingly, one embodiment of the methods of the invention comprises (a) contacting the biological sample with a plurality of different sized quantum dots formed in the cocktail solution by combining the two in solution, in which the sample is mixed with the cocktail solution; (b) waiting for a period such that the quantum dots are able to interact with the biological materials and bind with if an affinity exists; (c) passing the solution through the filter, which the bulk of the biological material, and any attached quantum dots remain trapped in; (d) analyzing the excess solution consisting of unbound quantum dots and activating those quantum dots to fluoresce using a light source and a detector capable of simultaneously detecting the presence of multiple quantum dots of different sizes.

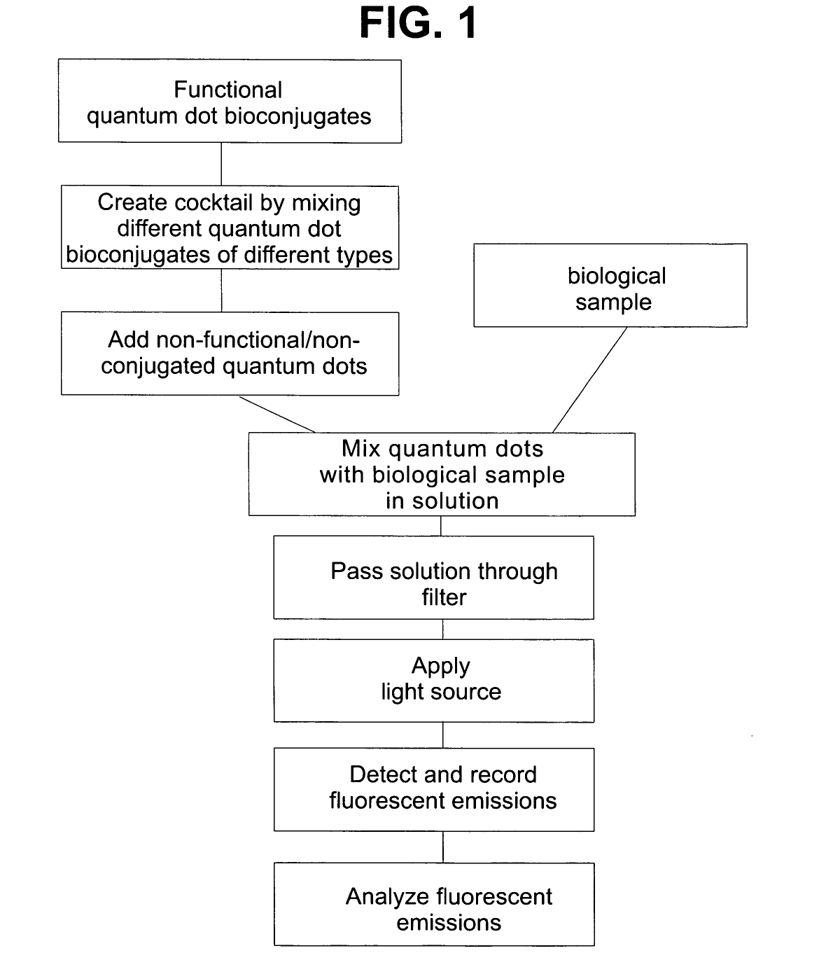

Operation of the embodiment of the invention is summarized in the steps shown in FIG. 1—Initially, quantum dots are created functional by bioconjugation with materials based on the experimental test to be performed on the biological material. Hence, if the presence of a specific type of cancer is to be tested for then the quantum dots will be functionalized with materials known to have an affinity specifically to cancerous cells. That way the presence, or at least absence, of cancerous cells can be verified by the binding of quantum dots conjugated with a functional material with known properties. There are various methodologies for conjugating materials to quantum dots and manufacturers, such methodologies for attaching materials to quantum dots are known by those skilled in the art.

Secondly, the bioconjugate/functional and non-functional/non-conjugate quantum dots are mixed in solution with a biological sample where the quantum dots are able to interact. If an affinity exists between the functional material present on the quantum dot bioconjugates and the biological material, binding will occur and the quantum dot will attach to the biological material. After a period of time lasting several seconds up to an hour the solution is then passed through the filter, where the unbound quantum dots will pass though due to the mesh or pore size of the filter being much greater than an individual unbound quantum dot. The biological material and any attached quantum dots present in the solution will trap in the filter, resulting in a final solution consisting of unbound quantum dots.

Finally by applying the light source either in a continuous or pulsing manner to excite the quantum dots into fluorescence, enables the detection of the different sized quantum dots based on their known fluorescent wavelengths. The fluorescent intensities and emissions from the activated quantum dots are then analyzed using computational software. From this, information about the biological material present in the sample can be deduced by correlating the quantum dot bioconjugates absent from the filtered solution by their conjugated functional material. Furthermore, the non-functional quantum dots used in the solution provide information on whether methods of the invention have been performed correctly and their presence in the final suspension provides experimental validation—their absence indicates whether the methods of the invention have been performed correctly or whether the results are inconclusive. The fluorescent intensity of the non-functional quantum dots also provides a calibration for the functional quantum dots present in the final, filtered solution—as there will inevitably be small quantities of all sizes of quantum dots present, information about the degree of interaction and binding of other quantum dots can be gathered by relative comparison of their intensities.

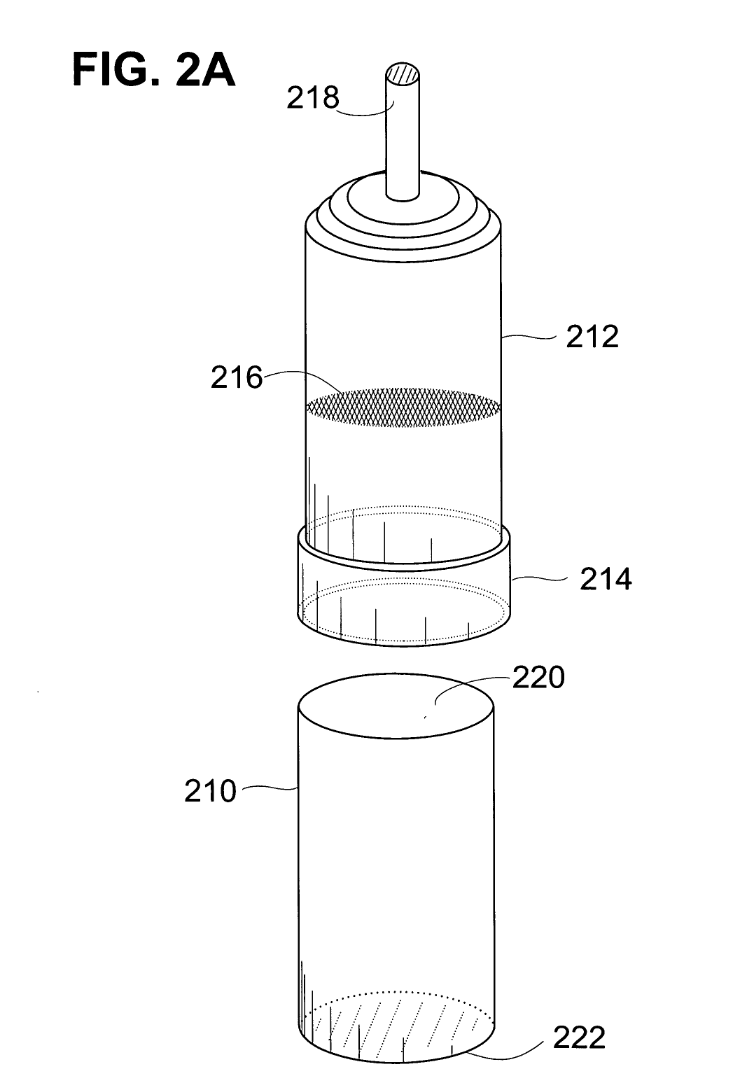

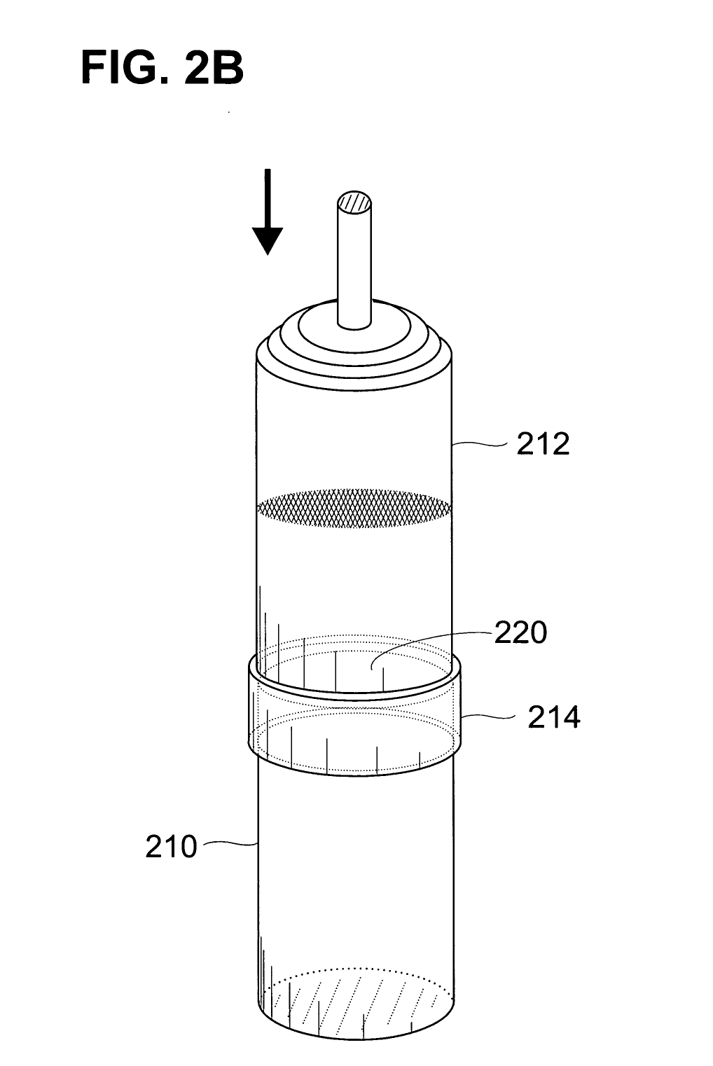

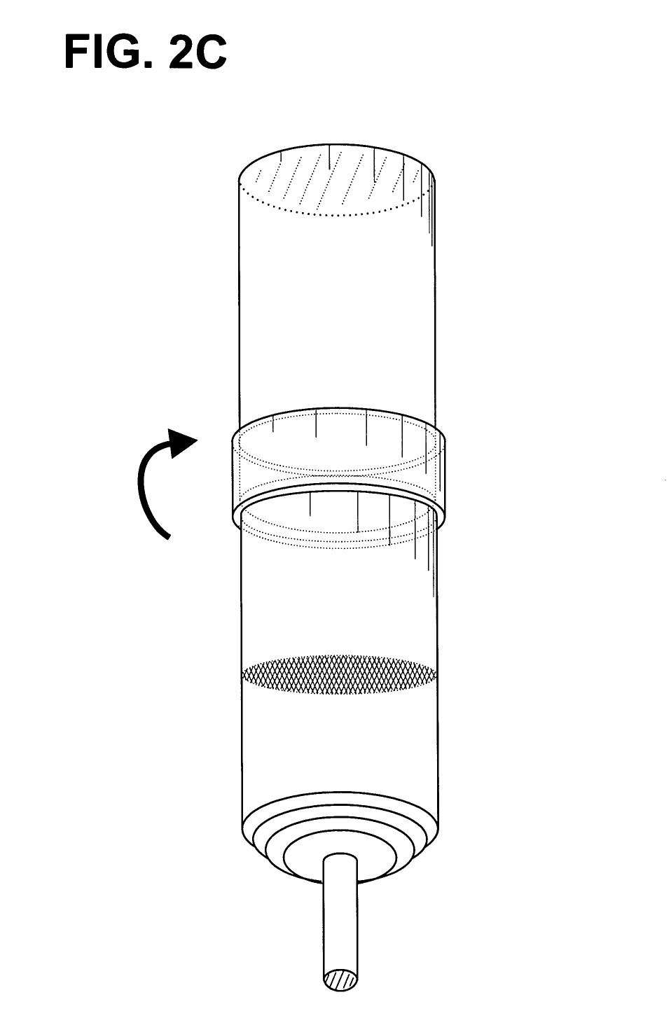

In view of the above, the present invention also provides an apparatus capable of performing the described methodology. One embodiment of the inventive apparatus is shown in FIG. 2A, which illustrates the use of a two-part, mix-and-flip apparatus for carrying out the methods of the invention. The term “mix-and-flip” is used herein to describe the operation in which the apparatus used; whereby the two-part, interconnecting tube system is inverted or flipped 180 degrees. Components of the apparatus used in the invention include a base tube 210 having an open 220 and a closed end 222 designed to adjoin to a filter tube 212, which houses an inbuilt mesh filter 216 and a closed nozzle 218 at one end. The mesh filter 216 is fixed and sealed at a position midway of the filter tube 212, and a connector 214 appended to the end of the filter tube 212 is used to connect the filter tube 212 to the base tube 210. In order to be able to detect fluorescent emissions from within the system it is a property that the filter tube 212, especially at the nozzle 218 be made from materials, such as plastic or glass, designed to be transparent at light in the 200-1100 nm wavelength range without loss.

As shown in FIG. 2B the filter 212 and the base tube 210 are designed to join together by connecting the connector 214 at the end of the filter tube 212 together with the open end 220 of the base tube 210, forming a sealed system, and ensuring nothing from inside the system can escape or leak, and likewise from the outside-in. Illustrating the flip phase of the operative methods used in the apparatus is shown in FIG. 2C where the interconnected system is inverted or flipped 180 degrees.

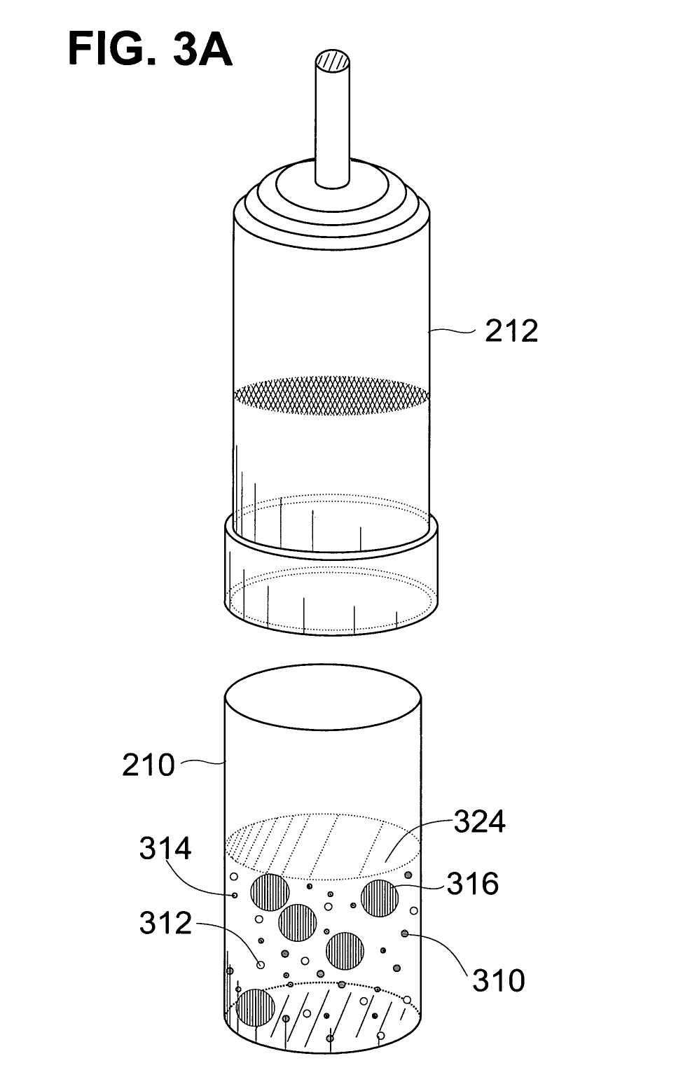

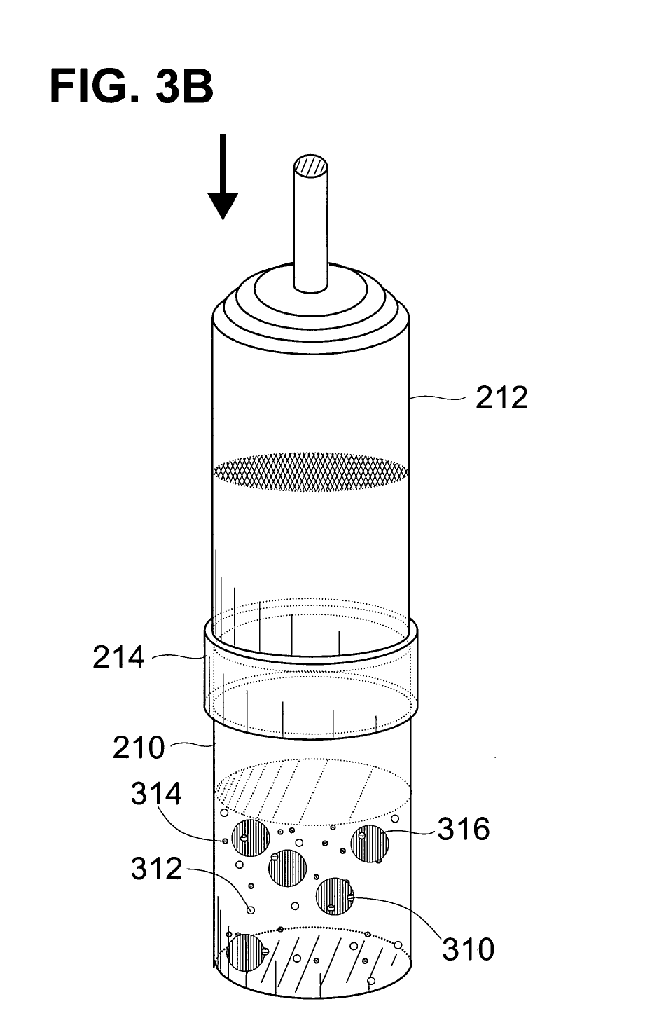

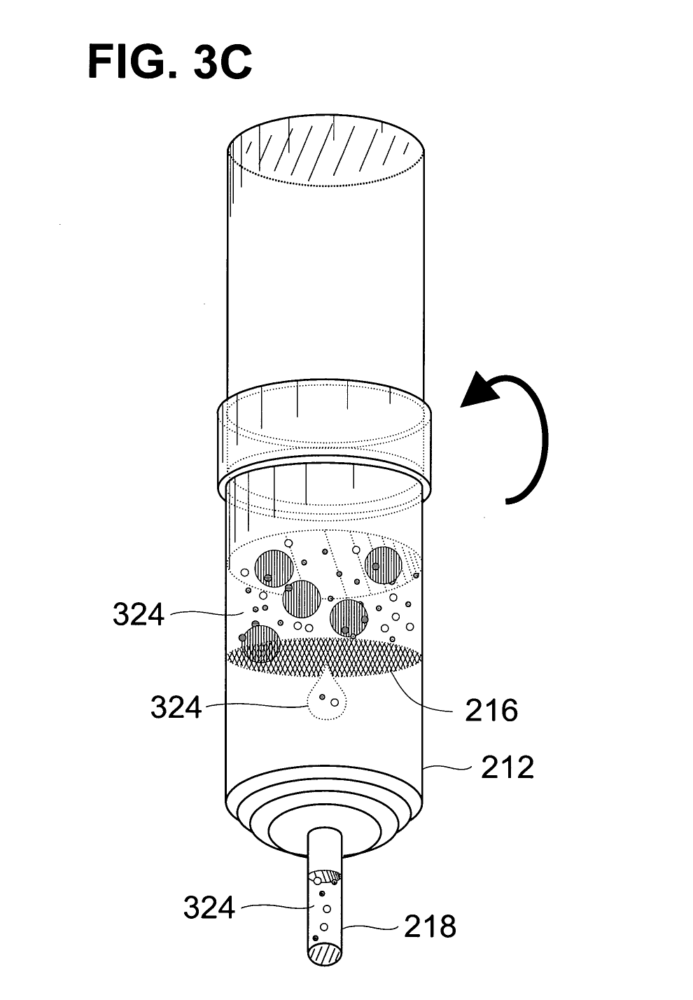

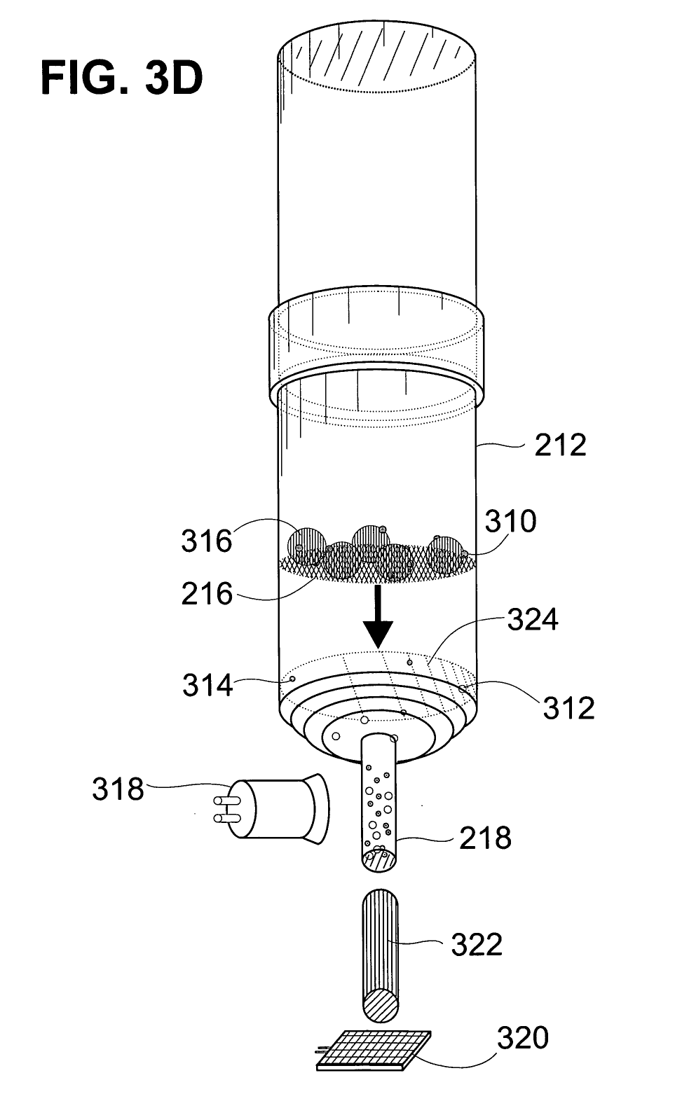

Practical operation of the mix-and-flip apparatus is illustrated sequentially in FIGS. 3A-D: Beginning with the perspective view shown in FIG. 3A, a solution 324 consisting of a plurality of 7 nm quantum dot bioconjugates 310; a plurality of non-functional 6 nm quantum dots 312; and a plurality of 5 nm quantum dots 314 functionalized with a different bioconjugate to that of 310 are mixed with the biological sample consisting of a plurality mammalian cells 316 in the base tube 210. As shown in FIG. 3B, immediately the filter tube 212 is connected to the base tube 210 to create a combined and sealed system. Gradually the functional 7 nm quantum dots 310 begin to bind to the mammalian cells 316 based on a positive affinity, whereas the non-functional 6 nm 312 and the 5 nm functional quantum dots 314 remain unbound due to no affinity.

As shown in FIG. 3C, after a period or time lasting several minutes to an hour, the combined system is inverted 180 degrees in what is known as the flip phase of operation. In the filtration phase- The solution 324 then comes in contact with the mesh filter 216 and begins to flow through and collect in the nozzle 218 at the base of the filter tube 212.

Finally, as shown in FIG. 3D, gravity forces the solution 324 to completely pass through the mesh filter 216, capturing the mammalian cells 316 and their attached functional 7 nm quantum dots 310. A light source 318 then activates the non-functional 6 nm 312 and the 5 nm functional quantum dots 314, which have passed through the mesh filter 216 and collected in the nozzle 218, and a fiber optical cable 322 is used to deliver the distinct fluorescent wavelengths to a detector 320. The experimental setup in FIG. 3D is configured for fluorescent transmission measurements with the detector 320 placed at a 90 degree angle with respect to the light source 318. The light source 318 is then pulsed (ie. Switched on and off periodically) and at intervals between the pulses the fluorescent emissions from the non-functional 6 nm 312 and the 5 nm functional quantum dots 314 are recorded by the detector 320. However, depending on whether recording absorbance or transmission fluorescent measurements, the light source 318 may be placed at a 180, or 90 degree angle to the detector 320, respectively. Even more, it is possible to do both absorbance and transmission fluorescence measurements by simultaneously collecting light at 90 and 180 degrees with respect to the light source 318. Hence, by recording the presence of quantum dots in the nozzle 218 it can be said by the absence of the 7 nm quantum dots bioconjugates 310 that the functional material present on those quantum dots has an affinity to the mammalian cells 318, and properties, traits and characteristics about those cells 318 can be made.

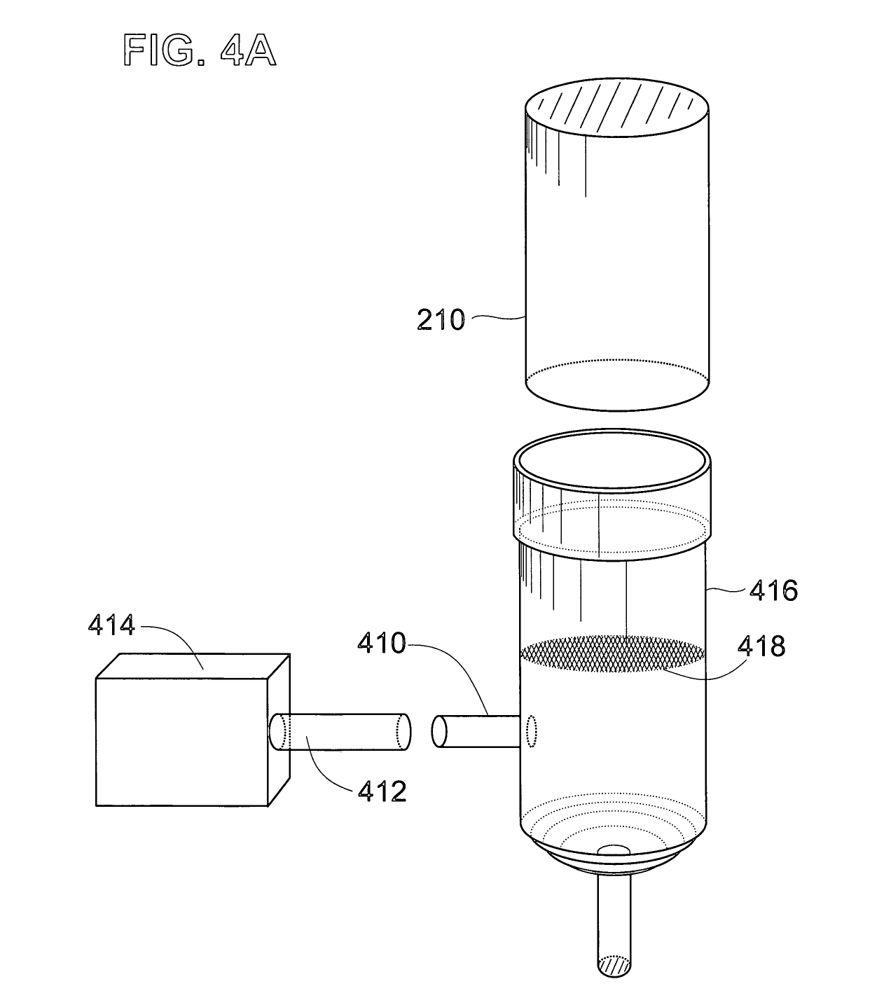

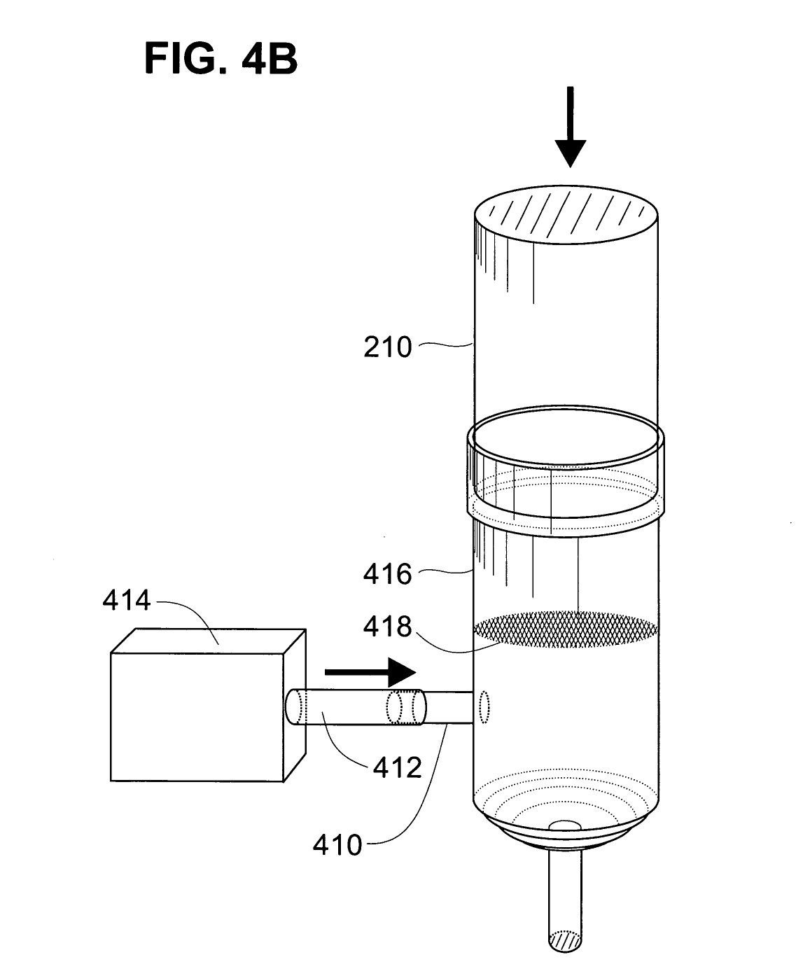

Another embodiment showing the addition of a suction outlet 410 and a pump 414 to the mix-and-flip apparatus used to perform the inventive methods is shown in FIG. 4A: Where the suction outlet 410 built below a pore filter 418 on an output-filter tube 416, and connected to an external pump 414 via a plastic tube 412 in order to create vacuum inside the output-filter tube 416. As shown in FIG. 4B the suction outlet 410 is connected to the external pump 414 via the attachment of the plastic tube 412, and the base tube 210 is joined with the output-tube 416 to create a sealed system. Advantage of this embodiment is realized when the biological material present in the sample has nanoscale dimensions, such as viruses, or in cases when large volumes require fast flow of the solution through the pore filter 418.

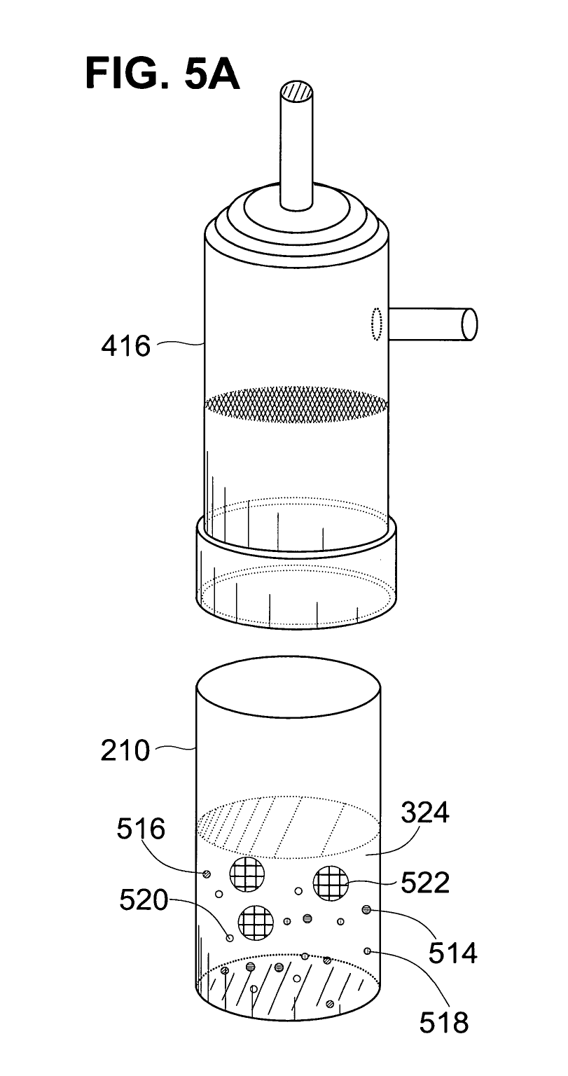

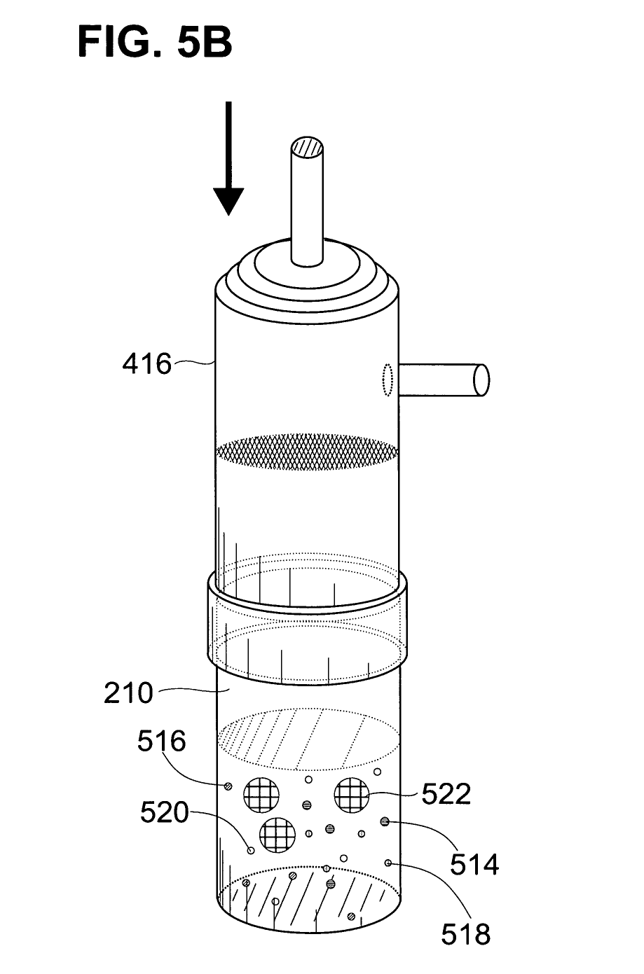

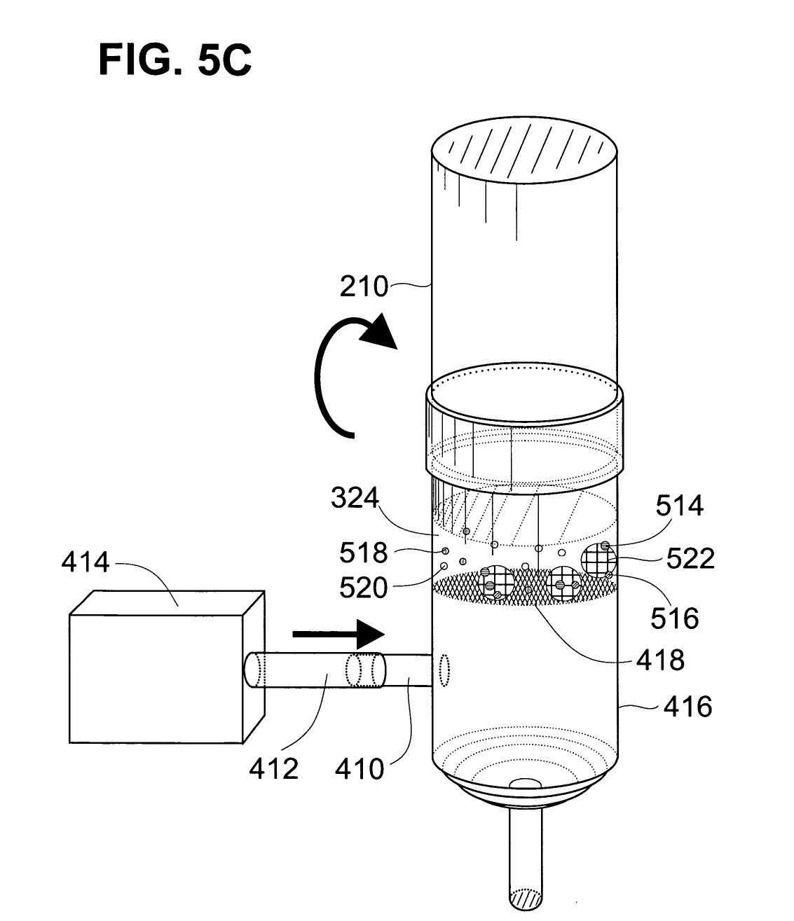

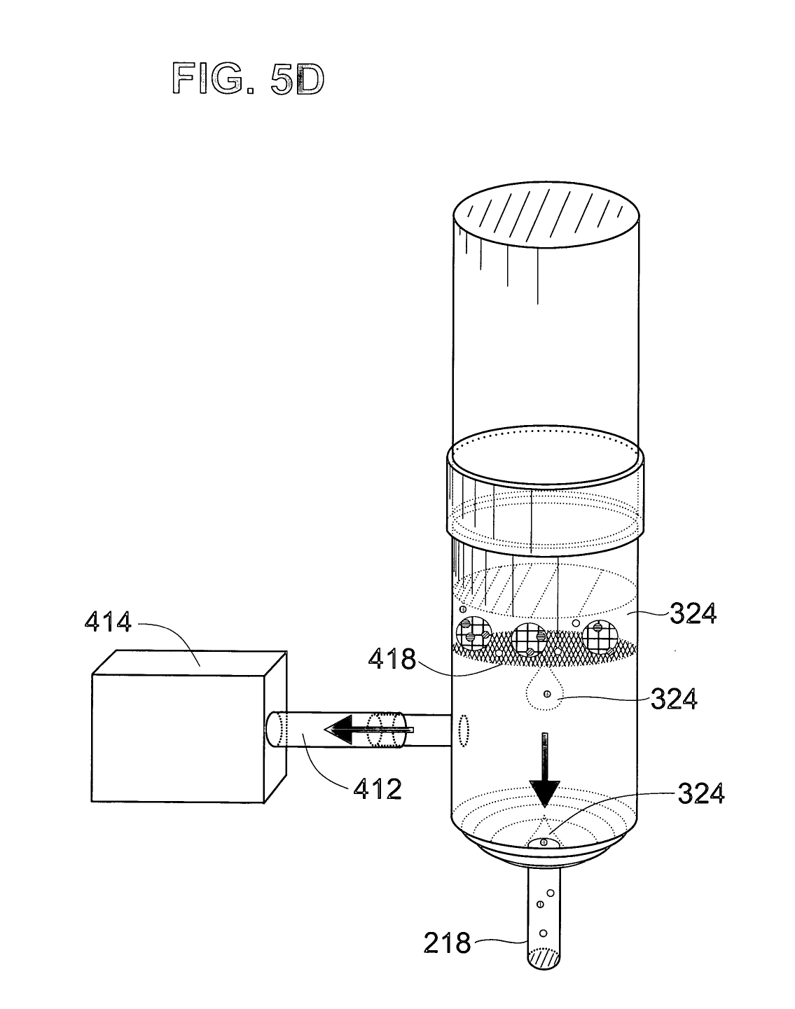

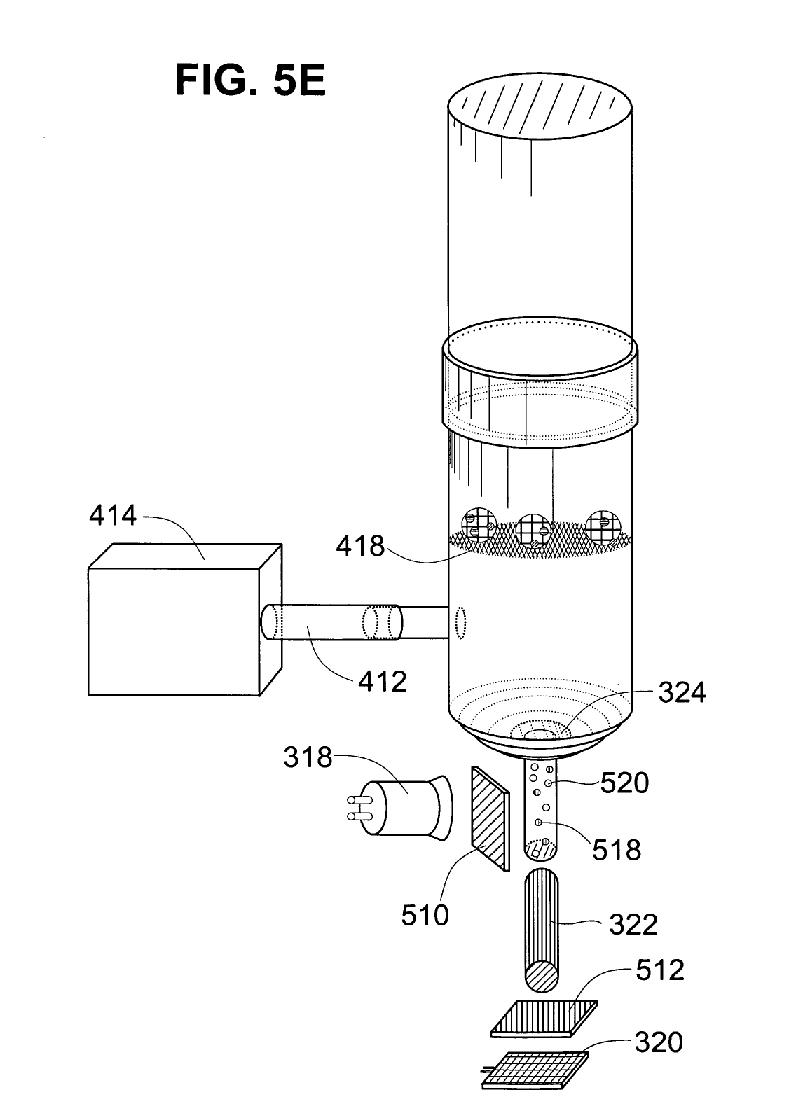

As shown in FIG. 5A operation of the apparatus begins with mixing a plurality of 10 nm 514, 9 nm 516 and 7 nm 518 quantum dot bioconjugates, with each size conjugated with different functional materials; and a plurality of 8 nm non-functional quantum dots 520 in the base tube 210 with a plurality of viral spores 522 in the solution 324. As shown in FIG. 5B, the base tube 210 is connected to the filter-output tube 416; as shown in FIG. 5C, the base 210 and the filter-output 416 tubes are then inverted and the plastic tubing 412 is connected to the suction outlet 410 on the filter-output tube 416 and to the pump 414. The solution 324 remains suspended above the pore filter 418 due to the small pore sizes used in the filter 418 to trap the viral spores 522. The 10 nm 514 and 9 nm 516 quantum dot bioconjugates begin to bind to the viral spores 522, with the 8 nm non-functional 520 and the 7 nm quantum dot bioconjugates 518 remaining unbound; as shown in FIG. 5D the pump 414 is turned on creating vacuum, which forces the solution 324 and the unbound quantum dots through the pore filter 418 and to collect in the nozzle 218 at the base of the filter-output tube 416.

As shown in FIG. 5E, when the solution 324 is completely drained through the pore filter 418 the pump 414 is turned off. To illustrate how light filters may be employed as a method in the apparatus—A low-pass light filter 510 is used to filter the light from the light source 318, and a band-pass light filter 512 is used to filter the fluorescent light from the 8 nm non-functional 520 and the 7 nm quantum dot bioconjugates 518 delivered by the optical fiber 322 to the detector 320. Using this setup the light source 318 can be applied while simultaneously detecting the fluorescent emissions.

The present invention has applications in various in vitro diagnostic assays, including, but not limited to, the detection of cancer, viruses, diseases and infections. The methods and apparatus of this invention can be used to study and profile a large number of genes, drug targets, and proteins, from a biological sample. Usefulness of the invention is particular realized when there is a very small tissue specimen or quantity of biological sample available for diagnosis, as effectively, the methodology used in this invention requires only a small amount of biological material.

### EXAMPLES

The present invention is described in further detail using the following examples. These examples are used to illustrate the apparatus and methods of the present invention and are not intended to limit the scope of the invention.

### Example 1

This example describes an instance outlining a potential application for the novel methods and apparatus described in the invention. In this case the aim is to examine the types, and quantities of hormones secreted from a biological sample containing a plurality of pituitary cells; a cell type known to secrete several hormone types. The cocktail solution contains several functional different sized quantum dots bioconjugated with hormone antibodies of anti-luteini sing (anti-LH), anti-follicle-stimulating hormone (anti-FSH), anti-growth hormone (anti-GH), anti-adrenocorticotropic hormone (anti-ACTH), anti-alpha melanocyte-stimulating hormone (α-MSH), anti-prolactin (anti-PRL), and anti-thyroid stimulating hormone (anti-TSH) as well as a unique size of non-functional quantum dots to act as the control. A supplement of potassium chloride at 5 mM concentration is added to the solution consisting of the quantum dots and the pituitary cells in order to induce widespread stimulation in the cell types present, and testing is performed using described methodology and apparatus given in the invention.

### Comparative Example 1a

This case highlights an instance of where it might be advantageous to only a single quantum dot bioconjugate in addition to the non-functional quantum dots used as the control- Instead of combining quantum dots with different functionalities, a single functional quantum dot conjugate with anti-luteinising hormone is used in addition to a different sized non-functional quantum dot to act as the control. The aim of this experiment is to determine the amount of luteinising hormone (LH) secreted in pituitary cells. In order to induce the secretion of this specific type of hormone, gonadotropin-releasing hormone (GnRH), which is known to those skilled in the art, to stimulate cells to secrete LH, is used as a supplement. Using the relative intensities of quantum dots in several tests introducing stimuli at different concentrations from those absent of stimuli, a relative comparison of the amount of quantum dots from the intensity in the final solution is made by employing the inventive methods and apparatus described in this invention.

### Example 2

In yet another example, a biological sample in the form of biopsy is taken from a patient with a tumor suspected of being malignant. While cancerous cells have different mutations, which make identification based on a single trait difficult, a cocktail solution of different sized quantum dots are functionalized with the dominant traits of cancers cells, such as epidermal growth factor receptor (EGFR) and insulin growth factor 1 receptor (IGF1R), which are widely recognized as validated cancer therapy targets. This allows testing for known characteristics of cancer cells simultaneously. Hence, different sized quantum dots bioconjugates are made from monoclonal antibodies designed to detect the alpha-subunits of IGF-1R and EGFR and used in the inventive methods and apparatus described in the invention.

I claim:

1. A method for testing a biological material, comprising:
(a) a plurality of quantum dot bioconjugates,
(b) a filter designed to trap said biological material,
(c) a light source for activating said quantum dot bioconjugates,
(d) a detector for recording fluorescence of said quantum dot bioconjugates,
(e) said biological material is mixed with said quantum dot bioconjugates in a solution,
(f) said solution is filtered through said filter,
(g) said quantum dot bioconjugates attached to said biological material will capture in said filter during filtration,
(h) said quantum dot bioconjugates not attached to said biological material will pass through said filter,
whereby properties of said biological material can be deduced by from the interaction of the quantum dot bioconjugates and sorted using said filter.

2. The method of claim 1 wherein a plurality of supplements is added to said solution,

3. The method of claim 1 wherein said filter is mounted in a tube.

4. The method of claim 1 wherein said quantum dot bioconjugates are of a plurality of different sizes.

5. The method of claim 4 wherein each size of said quantum dot bioconjugates are conjugated with a functional material different from the other size.

6. The method of claim 4 wherein each size of said quantum dot bioconjugates fluoresce at a peak wavelength not imposed by others of said different sizes.

7. The method of claim 1 wherein said solution is filtered through said filter using a pump.

8. The method of claim 1 wherein a plurality of non-functional quantum dots is added to said solution.

9. A method for identifying the characteristics of a biological material using a quantum dot cocktail, comprising:
(a) said quantum dot cocktail comprises of a plurality of quantum dots,
(b) said quantum dots are of a plurality of different sizes,
(c) said quantum dots are designed to interact with said biological material by mixing in a solution,
(d) a filter designed to capture said biological material and said quantum dots bound to and pass said quantum dots not bound to said biological material,

10. The method of claim 9 wherein said quantum dots of said different sizes are conjugated with a plurality of functional materials.

11. The method of claim 9 wherein at least one size of said quantum dots present in said solution remains non-functional.

12. An inter-connectable apparatus for separating a solution consisting of a plurality of unbound quantum dots from a plurality of bound quantum dots attached to a plurality of biological material, comprising:
(a) a filter tube with a filter mounted inside,
(b) a base tube for mixing the quantum dots with said biological material,
(c) said filter tube is designed to connect with said base tube and be inverted 180 degrees,
(d) said filter captures said biological material and said bound quantum dots and pass said unbound quantum dots, which is operated such that when said filter tube is connected with said base tube and inverted 180 degrees said solution passes through said filter.
(e) a light source for exciting said unbound quantum dots to fluoresce,
(f) a detector for recording fluorescent light emitted from said unbound quantum dots.

13. The apparatus of claim 12 wherein a suction outlet on said filter tube is connected to a pump and said solution is filtered through said filter using vacuum suction generated by said pump.

14. The apparatus of claim 12 wherein a low-pass optical filter is used to filter excitation light from said light source.

15. The apparatus of claim 12 wherein a band-pass optical filter is used to filter fluorescent emission from said unbound quantum dots to said detector.

16. The apparatus of claim 12 wherein an optical fiber is used to deliver excitation light dots from said light source to said unbound quantum dots.

17. The apparatus of claim 12 wherein said optical fiber is used to deliver fluorescent emissions from said unbound quantum dots to said detector.

18. The apparatus of claim 12 wherein said unbound quantum dots are collected in a nozzle on the end of said filter tube.

19. The apparatus of claim 12 wherein said unbound quantum dots and said bound quantum dots are of different sizes.
